(19) United States
(12) Patent Application Publication
(10) Pub. No.: US 2013/0006120 A1
(43) Pub. Date: Jan. 3, 2013

(54) MARKER FOR A MEDICAL NAVIGATION SYSTEM WITH A LASER TRACKER

(76) Inventors: Alexander Druse, Munich (DE); Alexander Urban, Forstinning (DE); Ingmar Thiemann, Munich (DE)

(21) Appl. No.: 13/635,079

(22) PCT Filed: Mar. 17, 2010
(86) PCT No.: PCT/EP2010/053421

§ 371 Date: Sep. 14, 2012

(51) Int. Cl. A61B 6/00

(52) U.S. Cl. 600/476

(57) ABSTRACT

A medical navigation system, comprising at least one laser scanner, at least one marker and a processing unit, wherein a laser scanner comprises a laser for generating a laser beam and a laser positioning unit for varying the direction of the laser beam in at least one dimension, wherein a marker comprises a detector for the laser beam and is configured to send an information signal to the processing unit when the laser beam hits the detector, and wherein the processing unit is configured to calculate the location of a marker from the information signal sent by the marker.

The present invention relates to a marker for a medical navigation system, a medical navigation system comprising a marker and at least one laser scanner, a method for determining the location of a marker and a software program for performing the method.

Medical navigation systems for determining the location of body structures or medical items such as surgical tools are found in the prior art and are widely known. The object to be located is provided with at least one marker which can be located by the medical navigation system. The most common configuration for determining the position of markers is for light to be emitted and for the light reflected by the markers to be detected using a stereoscopic camera. The drawback of this approach is that other structures might reflect the light onto the stereoscopic camera, in addition to the markers. It is therefore necessary to determine whether a reflection in the stereoscopic image has been caused by a marker or not.

It is an object of the present invention to provide a marker and a medical navigation system which reduce or avoid the disadvantages of the prior art.

This object is achieved by the marker of claim 1, the medical navigation system of claim 6, the method for determining the location of a marker according to claim 14 and the program according to claim 15. The invention also relates to a marker for a medical navigation system according to claim 4. Advantageous embodiments are specified in the dependent claims.

The marker according to the present invention comprises a detector for a laser beam and a device for sending an information signal to a processing unit of a medical navigation system when the laser beam hits the detector. The information signal can be sent immediately at the time the laser beams hits the detector or at a later point in time. Using this marker, it is possible to inform the processing unit when a laser which is scanning an area hits the marker. The processing unit can then determine the location of the marker from this information. A detailed description of this process will be given below.

Optionally, the marker has a unique ID and is configured to send its ID to the processing unit in the information signal. The processing unit can then unambiguously identify which marker has been hit by a laser beam.

The detector for the laser beam can for example be a photodetector, such as a photodiode, or a CCD chip.

In one embodiment, the marker comprises a device for determining the direction of impact of the laser beam. For example, the orientation of the marker can be determined from the direction of impact of the incident laser beam. The device for determining the direction of impact can comprise a plurality of photodetectors or a CCD chip. If a plurality of photodetectors are provided, each photodetector covers a particular sector. The direction of impact can be determined from the photodetector or the photosensitive element of the CCD chip which is hit. Preferably, the marker comprises a lens, in particular a wide-angle lens, positioned over the laser detector, in particular the CCD chip.

Additionally or alternatively, a marker comprises more than one detector for detecting a laser beam. If more than one detector is provided, a larger space surrounding the marker can be covered, which means that there are fewer blind spots from which an incident laser beam cannot be detected. The direction of impact of a laser beam can also be determined, or determined more exactly, using more than one detector.

The medical navigation system according to the present invention comprises at least one laser scanner, at least one marker as described above and a processing unit. A laser scanner comprises a laser for generating a laser beam and a laser positioning unit for varying the direction of the laser beam in at least one dimension. The processing unit is configured to calculate the location of the marker from the information signal sent by the marker. In this document, “calculating (or determining) the location” of a marker or an object in general is used synonymously with “locating” the marker or object. The term “location” means a point in space, described for example by coordinates. The location of a marker is calculated in relation to a reference such as for example a reference coordinate system.

Using this configuration, the laser positioning unit varies the direction of the laser beam over time, such that the beam describes a path or pattern. The marker reports the point in time at which the laser beam hits the marker to the processing unit. From the known direction of the laser beam at this particular point in time, the processing unit can determine, i.e. calculate, a straight line on which the marker is located.

In the simplest embodiment, the laser positioning unit varies the direction of the laser beam in only one and exactly one dimension. The laser beam pattern is then a straight line. In a more elaborate embodiment, the laser positioning unit is configured to vary the direction of the laser beam in two dimensions. These two dimensions can in particular be perpendicular to each other. Given this configuration of the laser positioning unit, the laser scanner can scan according to any two-dimensional pattern. Advantageous patterns are a zig-zag pattern and a line-by-line pattern. In the case of a line-by-line pattern, the laser beam scans a one-dimensional line, for example once or repeatedly, and then scans the next, parallel one-dimensional line. However, any suitable laser beam pattern can be implemented. A single run through a pattern is called a sweep.

The direction is preferably described by one or more angles. The direction is made known to the processing unit by storing appropriate information in the processing unit, for example in the form of a table or a function, describing the position of each laser beam over time. Alternatively, the processing unit can interrogate a laser scanner as to the direction of its laser beam at a particular point in time. Additionally or alternatively, the direction of the laser beam is encoded in the laser beam. For example, the frequency of the laser light increases or decreases continuously during each sweep, such that the current frequency of the laser beam reflects the current point within the sweep. The direction can also for example be encoded in a pulse sequence of the laser or can be modulated onto the laser.

In a preferred embodiment of the laser scanner, the laser source is stationary and the direction of the laser beam is varied by deflecting the laser beam, for example using a mirror or a crystal. In an alternative embodiment, the orientation of the laser source can be varied, thereby varying the direction of the laser beam.

If a single laser scanner is provided, the medical navigation system can only unambiguously locate a marker under certain circumstances, such as when there is a limited number or range of possible locations. In order to allow a marker to be located at an arbitrary three-dimensional location, the medical navigation system preferably comprises at least two laser scanners. The laser scanners are preferably displaced, such that there is a significant distance between them. The distance between the two laser scanners is preferably several times the size of a marker, for example at least 10 times, 50 times, 100 times, 200 times, 500 times or 1,000 times the size—for example, the diameter—of a marker. Preferably, the relative position between the laser scanners is known. In this document, the term “position” means a combination of the location, as described for example by spatial coordinates, and the orientation, i.e. the rotational alignment. The term “relative position” therefore means the distance and the relative alignment between two laser scanners.

If the processing unit knows the direction in which a marker is located in relation to each of the at least two laser scanners, then the three-dimensional location of the marker can be unambiguously calculated as the intersection of the directions, wherein the directions are straight lines originating from the laser scanners, for example originating from laser deflecting units.

If two or more laser scanners are used, the reliability and accuracy of the determined marker location can be increased. Compared to conventional stereoscopic cameras, a plurality of laser scanners is much easier and computationally less demanding to use than a plurality of different cameras.

In accordance with the present invention, two or more laser scanners can share the same laser source. The laser beam generated by a laser can for example be split into two or more beams, wherein each beam is deflected by a laser deflecting unit of a laser scanner.

Optionally, at least one laser scanner comprises a laser detector and a device for sending an information signal to the processing unit when a laser beam hits the detector. Using this configuration, the processing unit can determine the direction in which a second laser scanner is located relative to a first laser scanner. This is important for determining the relative position of the laser scanners, for self-calibration by the medical navigation system.

If a pair of laser scanners both comprise a laser detector and are configured to send an information signal to the processing unit when a laser beam hits the detector, the direction and the orientation of the second laser scanner relative to the first laser scanner can be determined by the processing unit.

In yet another embodiment, at least one laser scanner comprises means for taking a distance measurement. For example, the distance between two laser scanners which form a pair of laser scanners can be measured. Using this additional information, it is possible to determine the complete relative position, i.e. the combination of the relative location and the relative orientation, of the two laser scanners. A fully automatic calibration of the medical navigation system, in particular of the laser scanners, can then be carried out. It is also possible to measure the distance between the laser scanner and a marker.

Optionally, the laser detector on a laser scanner can be used to periodically calibrate another laser scanner. In each sweep of a first laser scanner, for example, the laser beam hits the laser detector of a second laser scanner. If there is a difference between the direction in which the second laser scanner is detected and the expected direction, the first laser scanner—in particular, the laser positioning unit of the first laser scanner—can be re-calibrated. Additionally or alternatively, a marker as described above can be placed in a fixed and known location. A “known location” means that the processing unit knows the location, and preferably the position, of the marker, for example in relation to the reference as described above. The marker can be used for calibration in the same way as the laser detector on a laser scanner. It might be also possible to calibrate the direction of the scanners through additional information, for example received from sensors like gyro-sensors. Also a fully automatic arrangement of the scanners might be possible, e.g. the scanners might be mounted on a motorized platform. If stepping motors are used for the motorized platform, the feedback from the stepping motors can optionally be used for calibration.

When more than one laser scanner is used, it is advantageous for the processing unit to know which laser, i.e. the laser beam of which laser scanner, hit or is hitting the marker. In a preferred embodiment, each laser has a unique ID which is encoded in the laser beam emitted from the laser, and a marker is configured to detect the laser ID and to send it to the processing unit in the information signal. In this embodiment, the laser can be unambiguously identified by way of the ID encoded in the laser beam. The various possible ways of encoding the laser ID in the laser beam include using different wavelengths of the laser light, pulsing the laser beam in a particular sequence or modulating a code onto the laser beam.

In another (alternative or additional) embodiment, the respective lasers are identified by the chronological order in which a marker is hit. Accordingly, the laser scanners perform their sweeps for example in turn, i.e. a laser scanner only starts its sweep once the sweep of the previous laser scanner has been completed. Optionally, there is a pause before or after each iteration through all the laser scanners which for example lasts at least twice as long as the longest sweep. The processing unit can determine the beginning of a new iteration on the basis of this pause, which means it can identify the laser scanners. If the laser scanners perform their sweeps in turn, each sweep is performed in a known time slot. A particular laser scanner is assigned to each time slot.

Optionally, the time at which a laser beam hits the detector of a marker is transmitted in the information signal, for example as a time stamp. In this case, the point in time at which the laser beam hits the marker is determined in the marker and then included in the information signal. Additionally or alternatively, the processing unit determines the point in time at which a laser beam hits a marker from the time at which the processing unit receives the information signal. The processing unit is then preferably configured to consider the information signal run time when calculating the location of a marker. The information signal run time can include the processing time in the marker for detecting the laser beam, the processing time in the marker for forming and sending the information signal, the transmission time between the marker and the processing unit, the processing time for receiving and analyzing the information signal, or any combination of these factors.

A marker can for example be connected to the processing unit by a wireless link or a dedicated cable. If a dedicated cable is used, the marker can be identified by the port to which the dedicated cable is connected, and the step of sending the marker ID in the information signal can be omitted. The wireless link can employ any suitable technology, for example a Bluetooth connection. The information signal can also be created in the marker and stored in an RFID chip. This RFID chip can then be read by the processing unit. It is then particularly advantageous for the information signal to include the time at which a laser beam hit the marker, the ID of the laser which emitted the incident laser beam and the ID of the marker.

The present invention also relates to a method for determining the location of a marker of a medical navigation system. This method comprises the steps of varying the direction of the beam of a laser in at least one dimension; detecting the point in time at which the laser beam hits a marker; and calculating the location of the marker from the detected point in time and the direction of the laser beam at this point in time. The point in time at which the laser beam hits the detector of a marker is preferably determined from an information signal which is sent by the marker when a laser beam hits it. The method can be implemented using a medical navigation system as described above. The method can also comprise additional steps as described with reference to the means of the medical navigation system.

The method in accordance with the invention is in particular a data processing method. The data processing method is preferably performed using technical means, in particular a computer. The computer in particular comprises a processor and a memory in order to process the data, in particular electronically. The calculating steps described are in particular performed by a computer. Steps of determining or calculating are in particular steps of determining data within the framework of the technical data processing method, in particular within the framework of a program. A computer is in particular any kind of data processing device. A computer can be a device which is generally thought of as such, for example desktop PCs or notebooks or netbooks, etc., but can also be any programmable apparatus, such as a mobile phone or an embedded processor. In particular, a computer can comprise a system (network) of “sub-computers”, wherein each sub-computer represents a computer in its own right. A computer in particular comprises interfaces in order to receive data and/or to perform an analog-to-digital conversion.

The invention also relates to a program which, when running on a computer or when loaded onto a computer, instructs a laser scanner to vary the direction of a laser beam in at least one dimension and instructs a processing unit to receive an information signal which indicates that the laser beam has hit a marker and to calculate the location of the marker from the information signal received and/or to a program storage medium on which the program is stored and/or to a computer on which the program is running or into the memory of which the program is loaded and/or to a signal wave, in particular a digital signal wave, carrying information which represents the program. The program can perform additional steps of the method described above or can instruct components of the medical navigation system to perform additional steps of the method described above.

Computer program elements of the invention can be embodied in hardware and/or software (including firmware, resident software, micro-code, etc.). The computer program elements of the invention can take the form of a computer program product which can be embodied by a computer-usable or computer-readable storage medium comprising computer-usable or computer-readable program instructions, “code” or a “computer program” embodied in said medium for use by or in connection with the instruction executing system. Such a system can be a computer; a computer can be a data processing device comprising means for executing the computer program elements and/or the program in accordance with the invention. Within the context of this application, a computer-usable or computer-readable medium is any medium which can contain, store, communicate, propagate or transfer the program for use by or in connection with the instruction executing system, apparatus or device. The computer-usable or computer-readable medium can for example be, but is not limited to, an electronic, magnetic, optical, electromagnetic, infrared or semiconductor system, apparatus, device or medium of propagation, such as for example the Internet. The computer-usable or computer-readable medium could even for example be paper or another suitable medium on which the program is printed, since the program could be electronically captured, for example by optically scanning the paper or other suitable medium, and then compiled, interpreted or otherwise processed in a suitable manner. The computer program product and any software and/or hardware described here form the various means for performing the functions of the invention in the example embodiment(s). The computer and/or data processing device can in particular constitute a guidance information device which includes means for outputting an item of guidance information. The item of guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or vibration element incorporated in an instrument).

The present invention also relates to another marker for a medical navigation system, wherein it is possible to use this marker independently of the marker and the medical navigation system described above. The marker comprises means for detecting the directions in which other markers are located and a device for transmitting the detected directions. A processing unit is configured to receive these directions from the marker. If a plurality or set of markers as described above is provided, the processing unit can calculate the relative positions of the markers. Depending on the number of markers in the set of markers, the required accuracy with which the directions in which the other markers are located are detected can vary. It can for example be sufficient to determine a sector in which a marker is located in relation to the determining marker. As the number of markers in the set of markers increases, so the accuracy with which the relative positions of the markers can be calculated also increases, even though the directions in which the other markers are located are only determined to a limited degree of accuracy in each marker.

The direction in which another marker is located is preferably detected using a beacon signal which is transmitted by said other marker. In general, a beacon signal is a signal which is broadcast such that a receiver can take a bearing of the beacon signal and therefore the beacon signal transmitter. This beacon signal can be an optical, acoustic or electromagnetic (radio) signal. Accordingly, the marker preferably comprises a beacon signal transmitting unit and a beacon signal receiving unit. The beacon signal receiving unit locates the other marker, i.e. the beacon signal receiving unit is the device for determining the direction in which the other marker is located.

Optionally, a detected direction is transmitted in an information signal. This information signal can contain additional information, for example the information described above with reference to the medical navigation system using laser scanners. A marker as described in this section can therefore be used in a medical navigation system as described above, if it contains a laser detector.

One example of a device for detecting the directions in which other markers are located is a photosensor, for example a photodiode. This type of detector is configured to detect light, for example in the visible or invisible spectrum, which falls on the marker and originates from a point within a limited sector of the space surrounding the marker. In another example, the device for detecting the directions in which other markers are located is a CCD chip. A lens, such as for example a wide-angle lens, is preferably positioned over the CCD chip. A CCD chip carries an array of photosensitive elements, for example a two-dimensional array. The direction of impact of the light can be determined by identifying the photosensitive element or elements which the incident light, for example in the visible or invisible spectrum, hits. A marker preferably also comprises a light source for emitting light, for example in the visible or invisible spectrum. The light emitted from the light source of the marker can be detected by one or more other markers.

If the device for detecting the directions in which other markers are located is an optical receiver such as a photosensor or a CCD chip, the location of the marker can be determined using a processing unit and at least one laser scanner as described above. In this case, the location of a marker can be determined relative to a reference coordinate system based for example on the positions of one or more laser scanners.

Other examples of devices for detecting the directions in which other markers are located are a set of coils, for example three coils arranged perpendicular to each other, or a plurality of antennas. Using the coils or the antennas, it is possible to determine the direction from which an electromagnetic signal originates, for example by signal processing. If such a device is used, the marker preferably comprises a device for transmitting electromagnetic signals, such as a coil or an antenna. Still other examples of devices for detecting the directions in which other markers are located are acoustic receivers for receiving an acoustic signal, for example in the audible or inaudible spectrum. In particular if a plurality of acoustic receivers are used, it is possible to determine the direction from which an acoustic signal originates, for example by signal processing. If acoustic receivers are used, the marker preferably comprises a device for transmitting acoustic signals, such as a speaker.

The device for transmitting the detected directions can for example be an optical, acoustic or electromagnetic transmitter. An optical transmitter can be a light source which for example emits light in the visible or invisible spectrum. An acoustic transmitter can be a speaker which for example emits acoustic waves in the audible or inaudible spectrum. The detected directions are then encoded into the optical signal transmitted by the optical transmitter or into the acoustic signal transmitted by the acoustic transmitter—for example, in an information signal. An electromagnetic transmitter transmits an electromagnetic signal in which the detected directions are encoded—for example, in an information signal. The electromagnetic transmitter can for example be a Bluetooth transmitter or an RFID chip. The information signal is for example stored in the RFID chip, which is then read by the processing unit.

In one embodiment of the invention, the device for transmitting the detected directions is the same device as the device for transmitting the signal which is used to detect the direction in which the emitting marker is located relative to the detecting marker.

The signal which is emitted by a marker and used to detect the direction in which this marker is located relative to a detecting marker, i.e. the beacon signal, preferably contains a unique ID of the transmitting marker. The ID can for example be contained in the information signal or represented by the wavelength of a transmitted optical, acoustic or electromagnetic signal. The ID can be assigned to the marker during an initialization process or from a built-in RFID chip. Additionally or alternatively, each marker is assigned a time slot, and the marker which is currently sending its beacon signal is identified by said time slot. Further specifics of this form of chronological identification can be those described above in relation to the identification of a laser scanner.

In one embodiment of the present invention, the marker comprises means for detecting the status of the marker. The status can for example include information about the temperature of the marker, a stain on the marker, the humidity and/or vibrations at or near the marker, or any combination of these factors. In a preferred variation of this embodiment, the means for transmitting the detected directions also transmits the status of the marker, for example in the information signal.

In one embodiment of the present invention, one marker within a set of markers is a master marker and the other markers within the set of markers are slave markers. In this embodiment, the slave markers transmit information, for example about the detected directions or their status, to the master marker which then relays the information to the processing unit. The distance between the master marker and the slave markers is then short, such that short-distance transmitters which have a low energy requirement can be used.

A marker according to the present invention can communicate with other similar or identical markers. By using this capability, the relative positions of the markers can be calculated without the means conventionally used for this purpose in a medical navigation system, such as for example a stereoscopic camera. The markers can also be used together with laser scanners or conventional devices such as a stereoscopic camera. The advantage of this is that markers which cannot be seen by the scanner or the camera, for example because they are obscured by other markers or structures, can still be located by the medical navigation system. The medical navigation system can also detect if a marker cannot be used, for example because it is contaminated, and can then use a back-up marker.

It is possible to automatically arrange different types of geometries. This means that the user is able to configure different marker geometries, e.g. a rectangle, triangle, hexagon and so on. This also might be an initial step during the startup, to arrange the different geometries automatically, e.g. group I flash 2 times group II flash 4 times. It is further possible to group different markers to different groups. This means that is possible to group different markers to one or more geometries, e.g. marker a is in group I while marker b is in group I but also group II. This might depend on which geometry is active.

In one embodiment, a plurality of markers are flexibly connected to each other, for example by one or more strings or the like or by being arranged on a flexible substrate such as a mask, a mesh or a piece of fabric having an appropriate shape. Such a flexible substrate can be easily applied to a patient. Any type or types of markers can be arranged on the flexible substrate, for example reflective markers such as are known from the prior art, markers comprising a laser detector or markers comprising means for detecting directions in which other markers are located.

It is within the scope of the present invention to combine two or more of the options described above and/or features from different embodiments, wherein one or more features of an embodiment can be implemented with or without one or more other features of said embodiment.

The invention shall be described in more detail below by means of example embodiments, by referring to the accompanying drawings. The figures show:

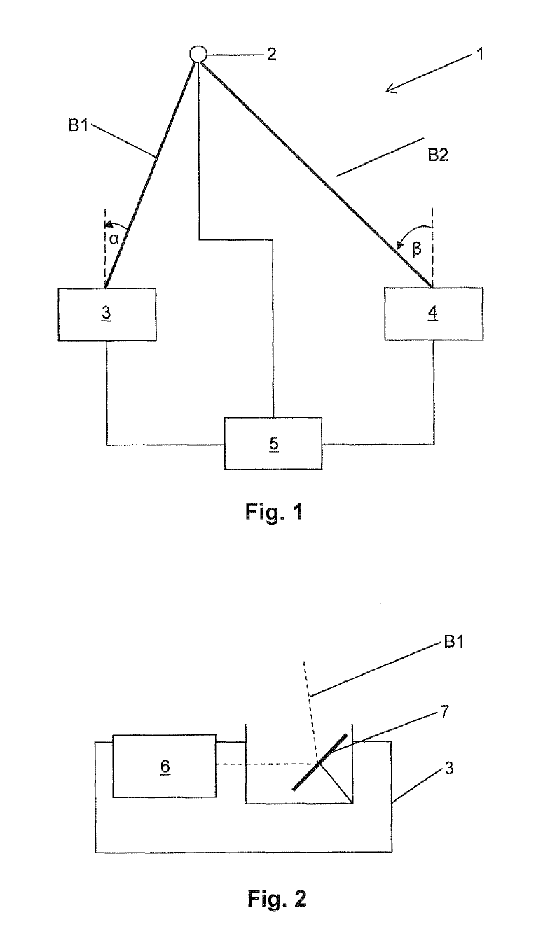

FIG. 1 a medical navigation system comprising two laser scanners;

FIG. 2 a laser scanner;

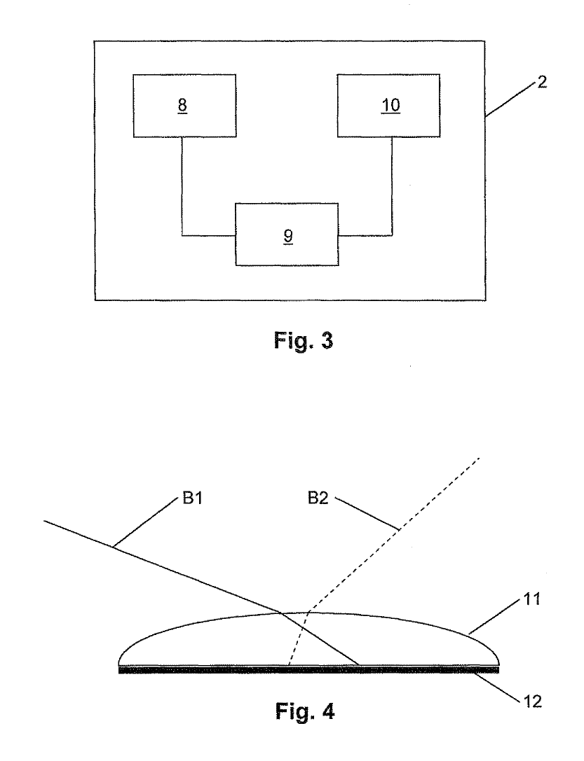

FIG. 3 a block diagram of a marker;

FIG. 4 a laser detector covered by a lens;

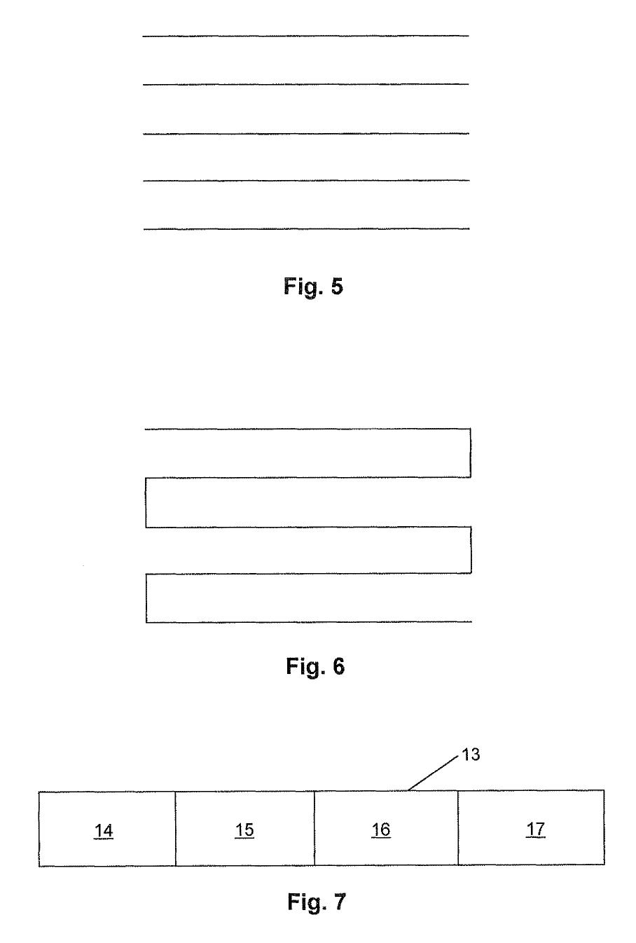

FIG. 5 a first scan pattern;

FIG. 6 a second scan pattern;

FIG. 7 a data structure of an information signal;

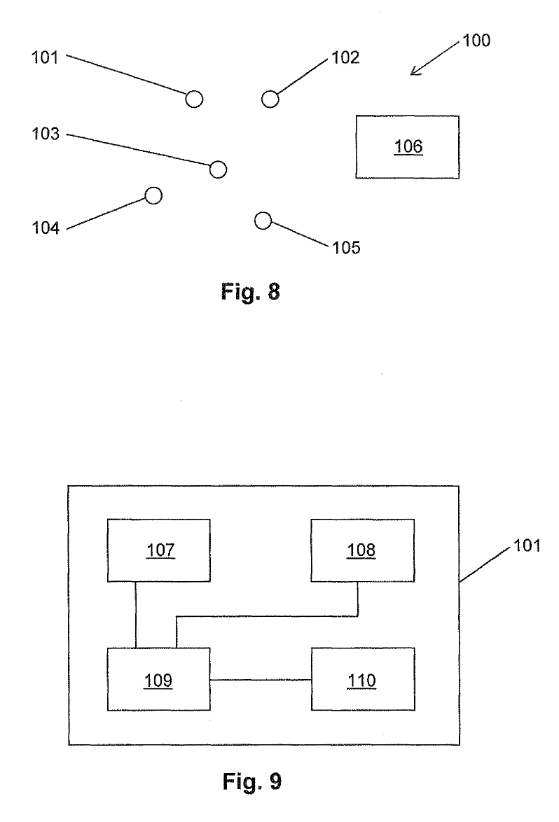

FIG. 8 a medical navigation system comprising communicating markers;

FIG. 9 a block diagram of a marker;

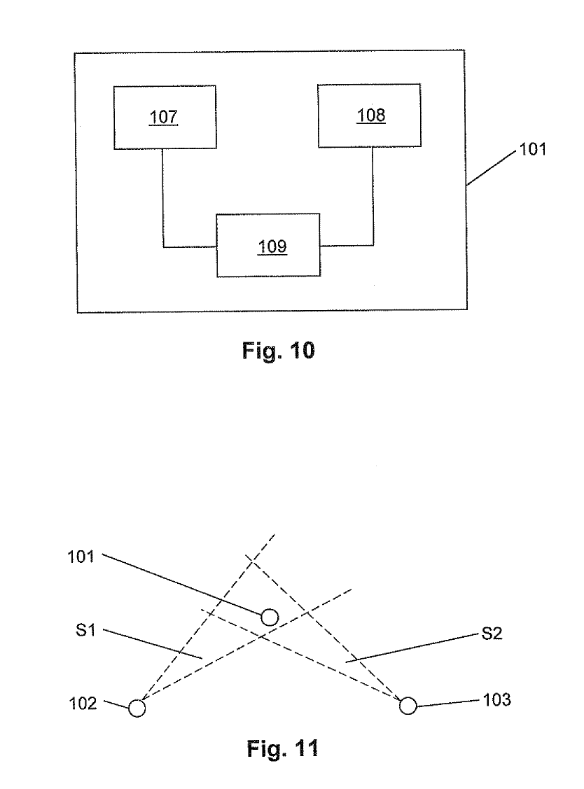

FIG. 10 a block diagram of a simplified marker;

FIG. 11 a representation of the principles of calculating a location; and

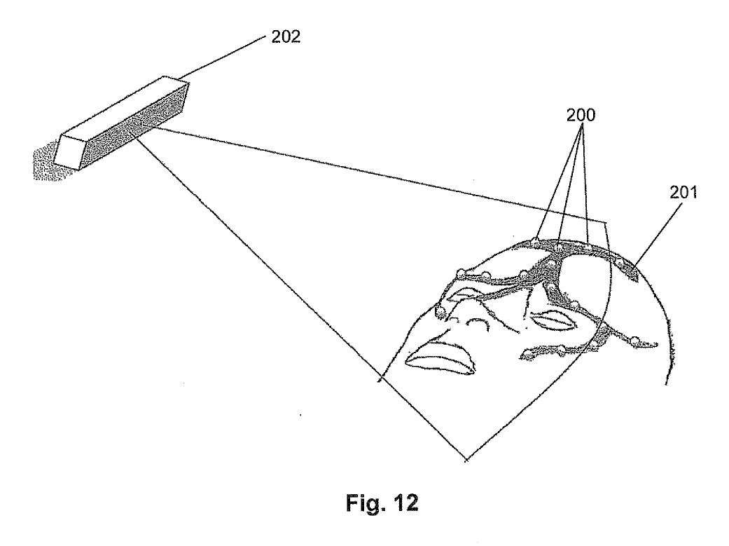

FIG. 12 markers on a flexible substrate.

FIG. 1 shows an example of a medical navigation system 1 comprising a marker 2, two laser scanners 3 and 4 and a processing unit 5. A possible structure of the laser scanners is shown in FIG. 2 by way of the example of the laser scanner 3. The laser scanners 3 and 4 can have the same structure.

As shown in FIG. 2, the laser scanner 3 comprises a laser 6 which generates a laser beam B1. The laser beam B1 is deflected by the deflecting unit 7 which is an example of a laser positioning unit and is capable of deflecting the laser beam B1 in one or preferably two dimensions. By being deflected, the laser beam B1 describes a given pattern. The pattern is drawn repeatedly, wherein each repetition is called a sweep. The laser deflecting unit 7 defines the angle or angles at which the laser beam leaves the laser scanner, i.e. it describes the direction in which the laser beam is emitted and the origin of the laser beam.

FIG. 5 schematically shows a preferred laser beam scan pattern. This scan pattern comprises a plurality of parallel lines. The laser beam scans each line one or more times, before scanning the next line and so on.

FIG. 6 schematically shows another preferred laser beam scan pattern. This pattern is a zig-zag pattern in which the laser beam proceeds from the upper left to the lower right in a meandering fashion, scanning one line from left to right and the next line from right to left, and so on.

The laser scanners 3 and 4 are connected to a processing unit 5 such that the processing unit 5 knows the directions in which the laser beams B1 and B2 of the laser scanners 3 and 4, respectively, are emitted. The processing unit 5 knows the directions of the laser beams B1 and B2 at each point in time, for example from a table or a function which describes a respective scan pattern, or can request the direction of a laser beam at a certain point in time from a laser scanner. The direction can also be encoded in the laser beam, decoded in the marker 2 and transmitted to the processing unit 5.

FIG. 3 schematically shows the structure of the marker 2. The marker 2 comprises a detector 8 for detecting a laser beam, a central processing unit (CPU) 9 and a transmitter 10. The detector 8 detects when a laser beam hits the marker 2 of one of the laser scanners 3 or 4. The detector 8 is connected to the CPU 9, such that the CPU 9 knows when a laser beam has hit the detector 8, and therefore the marker 2. When this occurs, the CPU 9 generates an information signal indicating that the marker 2 has been hit and sends the information signal to the processing unit 5 via the transmitter 10 in the marker 2. The transmitter 10 can be any kind of wireless transmitter, for example an optical, acoustic or radio transmitter. Examples of optical transmitters include light sources such as light emitting diodes. Examples of radio transmitters include Bluetooth transmitters or RFID chips. An example of an acoustic transmitter is a speaker. The transmitter 10 can alternatively be a transmitter for sending data via a cable.

The detector 8 can be any suitable detector such as for example a photodiode or a CCD chip. FIG. 4 shows an example of the detector 8 embodied as a CCD chip 12 which is covered by a lens 11. The lens 11 directs an incident beam of light onto a photosensitive element of the CCD chip 12 in accordance with the direction of impact of the incident beam. Using this configuration, the direction of impact of a laser beam can be determined and relayed to the processing unit 5 in the information signal.

Optionally, a filter is provided for the detector 8. This filter is adapted to block light of wavelengths other than those of the laser beams. This has the advantage that other light sources do not trigger the transmission of an information signal to the processing unit 5. The filter can be positioned over the lens 11, between the lens 11 and the detector 8 or within the lens 11. Another way of preventing an erroneous transmission of an information signal is to pulse or modulate the laser beam and to send the information signal only if the marker 2 detects a correct pulse or modulation scheme.

Calculating the location of the marker 2 is explained below by referring again to FIG. 1. For the sake of simplicity, the calculation explained is in two dimensions only. The location can however also be calculated in three dimensions in an analoguous way.

The laser scanner 3 emits a laser beam B1 and the laser scanner 4 emits a laser beam B2, each in a predefined pattern. When the laser beam B1 hits the marker 2, the marker 2 reports to the processing unit 5 that it has been hit by the laser beam B1 of the laser scanner 3 by sending an information signal to the processing unit 5. The processing unit 5 therefore knows the point in time at which the marker 2 was hit by the laser beam B1 and can determine the direction in which the laser beam B1 was emitted by the laser scanner 3 at that point in time. In the present example, this direction is indicated by an angle α in FIG. 1. Preferably, the processing unit 5 knows the signal run time, i.e. the period between the time at which the beam B1 hits the marker 2 and the time at which the processing unit 5 has analyzed the information signal.

In a similar way, the processing unit 5 determines the angle β as an indication of the direction in which the laser scanner 4 emitted the laser beam B2 at the point in time at which the beam B2 hit the marker 2. The position of the marker 2 is the point in space at which a straight line from the laser scanner 3 in the direction indicated by the angle α and a straight line from the laser scanner 4 in the direction indicated by the angle β intersect.

In the simplest embodiment, the marker 2 carries a simple photodiode which is connected to the processing unit 5 by a cable. A laser beam hitting the photodiode generates a voltage which is detected by the processing unit 5. In this embodiment, the CPU 9 and the transmitter 10 can be omitted. The photodiode is then the device for sending an information signal to the processing unit 5. If a plurality of markers is provided in the medical navigation system, the marker 2 can be identified by the hardware port of the processing unit 5 to which the marker is connected. In a more elaborate embodiment, the marker 2 sends an information signal to the processing unit 5, for example by a cable or via a wireless link.

FIG. 7 schematically shows an example of a data structure of an information signal 13 as sent by the marker 2. This structure comprises a marker ID field 14, a laser scanner ID field 15, a time stamp field 16, a direction of impact field 17 and a laser beam direction field 18, wherein each of the fields is optional depending on how the medical navigation system 1 is implemented. In a very basic implementation, the information signal can be a basic signal such as a dirac pulse or a logical signal which changes from high to low or vice versa.

The marker ID field 14 contains the ID of the marker which sends the information signal 13, i.e. the ID of the marker which has been hit. This ID can be hard-coded in the marker 2, for example in the central processing unit 9 or the transmitter 10. Alternatively, the marker ID can be assigned when the medical navigation system is calibrated. The marker ID field 14 can be omitted as applicable, for example if the marker 2 is connected to the processing unit 5 by a dedicated cable.

The laser scanner ID field 15 contains the ID of the laser scanner which emitted the laser beam which has hit the marker 2 and thus triggered the transmission of the information signal 13. The laser scanner ID can be encoded into the laser beam emitted by the laser scanner, for example in the wavelength of the laser beam, in a pulse sequence of the laser beam or by being modulated onto the laser beam. The laser scanner ID field 15 can be omitted as applicable, for example if the laser which emitted the beam which has hit the marker 2 can be identified in other ways, for example from the point in time at which or the time slot within which the marker 2 was hit.

The time stamp field 16 contains a time stamp which represents the point in time at which the marker 2 was hit by a laser beam. From this time stamp, the processing unit 5 can easily determine the directions in which the laser scanners emitted their respective laser beams at this point in time. If the time stamp is sent by the marker 2, differences in signal run time between the marker 2 and the processing unit 5 do not impair the calculated location of the marker. However, the time stamp field 16 can be omitted as applicable, for example if the signal run time is known.

The direction of impact field 17 contains information about the direction of impact of the laser beam which has hit the marker 2. This direction can for example be determined by a configuration using a CCD chip 12 and a lens 11, as described above with reference to FIG. 4. If the processing unit 5 knows the direction of impact, it can calculate not only the location but also the orientation of the marker 2.

The laser beam direction field 18 contains the direction in which the detected laser beam was emitted from the laser scanner. This is possible if the beam direction is encoded in the laser beam and decoded in the marker 2. The direction can for example be encoded in the laser beam by increasing or decreasing the frequency of the laser light during each sweep, by modulating the direction onto the laser beam or by encoding the direction into a pulse sequence of the laser beam.

The processing unit 5 knows the positions, i.e. the locations and orientations, of each of the laser scanners 3 and 4. These positions can be programmed into the processing unit 5 or can be automatically determined when the medical navigation system 1 is calibrated.

For automatic calibration, which is an advantage of the present invention, at least one and preferably all of the laser scanners comprise a detector for a laser beam. If three laser scanners are provided, the location of a laser scanner can be calculated in the same way as the location of a marker. Preferably, one of the laser scanners comprises means for taking a distance measurement, particularly if only two laser scanners are provided. The location of a second scanner relative to a first scanner can be calculated from the direction in which the second scanner is located relative to the first scanner and the measured distance. The relative orientation of a pair of scanners can be detected if the laser beam detector on a laser scanner is capable of detecting the direction of impact of the incident laser beam, as described with reference to FIG. 4, or if both laser scanners comprise a laser beam detector, which means that for each laser scanner, the processing unit 5 knows the direction in which the other laser scanner is located.

FIG. 8 shows a medical navigation system 100 comprising five communicating markers 101, 102, 103, 104 and 105 and a processing unit 106. The structure of the markers is shown in FIG. 9 by the example of the marker 101. Each marker comprises a detection unit 107, a central processing unit 109, a transmitter 108 and a beacon generator 110. The detector 107 is configured to detect the direction in which another marker is located by detecting the direction of impact of the beacon signal generated and broadcast by the beacon generator 110 of the other marker.

The beacon signal can be any optical, acoustic or electromagnetic (radio) signal. In a preferred embodiment, the beacon generator 110 is a light emitting diode. The detector 107 can for example be an arrangement of perpendicular coils, an antenna array or an optical or acoustic detector, depending on the type of beacon generator employed in the other markers. An optical detector can comprise a plurality of photosensitive elements, each responding to incident light from a certain direction or sector. The optical detector can for example be a CCD chip 12 in combination with the lens 11 as described with reference to FIG. 4.

The central processing unit 109 is configured to send the direction in which another marker is located to the other markers and/or to the processing unit 106 via the transmitter 108. The transmitter 108 can be any suitable transmitter, such as for example an optical, acoustic or electromagnetic transmitter such as a Bluetooth transmitter or an RFID chip.

FIG. 10 shows a simplified embodiment of a marker in which the transmitter 108 also performs the function of the beacon generator 110. This means that the transmitter 108 can transmit not only the directions in which the other markers are located but also a beacon signal which is used by another marker to detect the direction in which the transmitting marker is located. Preferably, the signal in which the detected direction of the other markers is transmitted is also the beacon signal.

The principle of calculating the relative locations of the markers 101 to 105 will now be described with reference to FIG. 11. FIG. 11 shows the markers 101, 102 and 103, wherein the marker 101 sends a beacon signal such as for example a beam of light. The marker 102 detects the beacon signal emitted by the marker 101 and determines that the marker 101 is located in the sector S1. The marker 103 detects the beacon signal emitted by the marker 101 and determines that the marker 101 is located in the sector S2. These two items of location information can be combined in order to determine that the marker 101 must be located in an area which is defined by the intersection of the sector S1 and the sector S2. Clearly, the accuracy of the detected location of the marker 101 can be increased by increasing the number of markers which detects the beacon signal emitted by the marker 101. There is no need to increase the detection accuracy of each detecting marker.

In a preferred embodiment, each marker 101 to 105 sends the direction in which the other markers are located to the processing unit 106 of the medical navigation system 100. The processing unit 106 then calculates the relative locations of the markers from these directions, as explained above. Each marker can send the detected directions directly to the processing unit 106. In an alternative embodiment, one of the markers is designated as a master marker and the other markers are designated as slave markers. The slave markers then send the detected directions to the master marker which in turn sends all the collected detected directions to the processing unit 106. Optionally, the processing unit 106 is located within the master marker.

The markers 101 to 105 can broadcast their respective beacon signals either simultaneously or sequentially. An ID for identifying the transmitting marker is preferably incorporated into the beacon signal, for example by pulsing or modulating the beacon signal or by choosing an appropriate frequency for the beacon signal. If the beacon signals are broadcast sequentially, then the markers can be identified by the time slot within which the beacon signal is broadcast.

The advantage of communicating markers is that a stereoscopic camera is not required, as in conventional medical navigation systems, because the markers themselves can collect all the information which is necessary in order to calculate the relative locations or positions of the markers. However, it is advantageous to use the communicating markers in combination with laser scanners as described with reference to FIGS. 1 to 7 or a conventional stereoscopic camera of a medical navigation system. The scanners or the camera can then detect the locations of the markers within the field of view of the scanners or the camera. Markers which are not within this field of view, for example because of an obstacle between the marker and the camera/scanner, can be located by using the other markers, and the navigation system can then combine the location information from the scanners or the stereoscopic camera and the location information from the communicating markers.

Optionally, a marker can comprise means for detecting the status of the marker, such as staining, temperature or movement and/or vibration. The marker is configured to transmit the status information to the processing unit 106 via the transmitter 108. The processing unit 106 can then evaluate the reliability of the marker.

FIG. 12 shows a multitude of markers 200 arranged on a flexible carrier 201. A sensing means 202 forms part of a medical navigation system and is designed to determine the locations of the markers 200. The sensing means 202 can be a laser scanner, and the markers 200 can be markers comprising laser beam detectors such as those described with reference to FIGS. 1 to 7. Alternatively, the sensing means 202 can be a stereoscopic camera. The markers 200 can be communicating markers 200 such as those described with reference to FIGS. 8 to 11. The stereoscopic camera 202 is then optional.

The flexible carrier 201 can be a mask made of a flexible material or flexible fabric. The carrier 201 can then adapt to the structure—in FIG. 12, the face of a patient—which is to be registered by the medical navigation system.

Using communicating markers as the markers 200 arranged on the flexible carrier 201 is particularly advantageous if the surface of an object is to be measured or surveyed. In this case, only the three-dimensional surface of an object is to be detected, and not its position in space. Therefore, the set of communicating markers can autonomously determine the relative positions of the markers and transmit the measurement results, for example in the form of a set of surface points.

1. A marker for a medical navigation system, comprising a detector for detecting a laser beam and a device for sending an information signal to a processing unit of the medical navigation system when the laser beam hits the detector wherein the marker has a unique ID and is configured to send its ID to the processing unit in the information signal.

2. (canceled)

3. The marker according to claim 1, wherein the marker comprises a device for determining the direction of impact of the laser beam.

4. A marker for a medical navigation system, according to claim 1, comprising a device for detecting directions in which other markers are located and a device for transmitting the detected directions.

5. The marker according to claim 4, wherein a marker comprises a beacon signal transmitting unit and a beacon signal receiving unit.

6. A medical navigation system, comprising at least one laser scanner, a processing unit and at least one marker comprising a detector for detecting a laser beam and a device for sending an information signal to the processing unit when the laser beam hits the detector wherein a laser scanner comprises a laser for generating a laser beam and a laser positioning unit for varying the direction of the laser beam in at least one dimension, and wherein the processing unit is configured to calculate the location of a marker from the information signal sent by the marker.

7. The medical navigation system according to claim 6, wherein a laser positioning unit is configured to vary the direction of the laser beam in two dimensions.

8. The medical navigation system according to claim 6, comprising at least two laser scanners, wherein at least one laser scanner comprises a laser detector and a device for sending an information signal to the processing unit when a laser beam hits the detector.

9. The medical navigation system according to claim 6, wherein at least one laser scanner comprises means for taking a distance measurement.

10. The medical navigation system according to claim 6 wherein a laser has a unique ID which is encoded into the laser beam emitted from the laser, and a marker is configured to detect the laser ID and to send it to the processing unit in the information signal.

11. The medical navigation system according to claim 6, wherein the processing unit is configured to consider the information signal run time when calculating the location of a marker.

12. The medical navigation system according to claim 6, wherein a laser scanner is configured to encode the direction of the laser beam into the laser beam.

13. The medical navigation system according to claim 6, comprising a marker according to claim 1 at a fixed and known location for calibrating a laser scanner.

14. A method for determining the location of a marker of a medical navigation system, comprising the steps of:
varying the direction of the beam of a laser in at least one dimension;
detecting the point in time at which the laser beam hits a marker; and
calculating the location of the marker from the detected point in time.

15. A program which, when running on a computer or when loaded onto a computer, instructs a laser scanner to vary the direction of a laser beam in at least one dimension and instructs a processing unit to receive an information signal which indicates that the laser beam has hit a marker and to calculate the location of the marker from the received information signal, and/or a program storage medium on which the program is stored, and/or a computer on which the program is running or into the memory of which the program is loaded.

16. The medical navigation system according to claim 1, comprising a marker according to claim 4 at a fixed and known location for calibrating a laser scanner.
